(19) United States
(12) Patent Application Publication
(10) Pub. No.: US 2026/0096985 A1
(43) Pub. Date: Apr. 9, 2026

(54) Injectable Composition For Drug Delivery

(71) Applicant: Celanese EVA Performance Polymers LLC, Irving, TX (US)

(72) Inventors: Vijay Gyanani, Powell, OH (US); Jeffrey Haley, Norwood, OH (US)

(21) Appl. No.: 19/348,865

(22) Filed: Oct. 3, 2025

Related U.S. Application Data
(60) Provisional application No. 63/704,049, filed on Oct. 7, 2024.
(60) Provisional application No. 63/824,296, filed on Jun. 16, 2025.

(51) Int. Cl. A61K 9/00; A61K 31/499; A61K 31/4995; A61K 31/519; A61K 31/551; A61K 31/554; A61K 47/34

(52) U.S. Cl. CPC A61K 9/0024; A61K 31/499; A61K 31/4995; A61K 31/519; A61K 31/551; A61K 31/554; A61K 47/34

(57) ABSTRACT

An injectable composition for delivering a therapeutic agent is provided. The injectable composition includes one or more polymers including a poly ortho ester (POE) polymer. The POE polymer has a T_{g} of between about −10° C. and 110° C. as determined in accordance with ASTM E1640-18. The injectable composition also includes one or more solvents and one or more therapeutic agents. The one or more therapeutic agents includes a basic therapeutic agent. Methods for treating a condition in a patient are also provided.

### RELATED APPLICATIONS

The present application is based upon and claims priority to U.S. Provisional Patent Application Ser. No. 63/704,049, having a filing date of Oct. 7, 2024, and U.S. Provisional Patent Application Ser. No. 63/824,296, having a filing date of Jun. 16, 2025, which are incorporated herein by reference.

### BACKGROUND OF THE DISCLOSURE

There are a number of diseases for which continued release of a drug or multiple drugs are required to treat and or ameliorate one or more causes or symptoms of the disease. Biodegradable polymers have been used for various medical applications including implants, suspensions, and other drug delivery systems. Often, biodegradable polymers are utilized in injectable formulations and can include microspheres or microcapsules. Injectable formulations avoid the need for an incision needed to implant a drug delivery system. However, manufacture, storage and injectability of microspheres or microcapsules formulations present problems. Further, providing therapeutically effective amounts of drugs often requires multiple injections, which can decrease patient compliance and clinical outcomes.

In light of these difficulties, a need continues to exist for a drug delivery system that is capable of delivering a therapeutic agent over a sustained period of time.

### SUMMARY OF THE DISCLOSURE

In accordance with one embodiment of the present disclosure, an injectable composition for delivery of a therapeutic agent is provided. The injectable composition includes one or more polymers including poly ortho ester (POE) polymer. The POE polymer has a T_{g} of between about −10° C. and 110° C. as determined in accordance with ASTM E1640-18. The injectable composition also includes one or more solvents and one or more therapeutic agents. The one or more therapeutic agents includes one or more basic therapeutic agents. Methods for treating conditions in a patient in need thereof are also provided.

Other features and aspects of the present disclosure are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended drawings in which:

FIG. 1 is a graph illustrating the percent release of risperidone from Comparative Example 1 and Examples 2-5.

### DETAILED DESCRIPTION

It is to be understood by one of ordinary skill in the art that the present discussion is a description of exemplary embodiments only and is not intended as limiting the broader aspects of the present disclosure.

Generally speaking, the present disclosure is directed to an injectable flowable composition that is capable of delivering one or more therapeutic agents that can prohibit and/or treat a condition, disease, and/or cosmetic state in a patient in need thereof (e.g., human, pet, farm animal, etc.). The injectable flowable composition includes a POE polymer having a glass transition temperature ranging from about −10° C. to 110° C. as determined in accordance with ASTM E1640-18 dispersed in one or more solvents. One or more therapeutic agents can be included in the injectable flowable composition. The POE polymer can have a molecular weight ranging from about 1 kDa to about 100 kDa, such as from about 20 kDa to about 60 kDa. The injectable flowable composition exhibits fluid-like properties such that it can easily be injected into a patient through the needle of a syringe, such as a standard 20 gauge needle.

Through selective control over the particular nature and concentration of the components of the injectable flowable composition, the present inventors have discovered that the resulting composition can be effective for sustained release of a therapeutic agent over a prolonged period of time. For instance, through selective control of the particular monomers used to form the POE polymer in combination with one or more basic therapeutic agents, the present inventors have discovered that the implants will not rapidly degrade and thus are suitable for providing longer term release of the drug within the patient before bio eroding. For example, the injectable flowable composition can release one or more therapeutic agents for a time period of about 3 days or more, in some embodiments, 5 days or more, in some embodiments about 10 days or more, in some embodiments from about 20 days to about 210 days, and in some embodiments, from about 30 days to about 180 days.

Various embodiments of the present disclosure will now be described in more detail.

### I. Injectable Flowable Composition

### A. Polymer

As indicated above, the injectable composition contains one or more polymers. Suitable polymers can include a hydrophobic polymer that can retain its structural integrity for a certain period of time when placed in an aqueous environment, such as the body of a mammal, and stable enough to be stored for an extended period before use. The hydrophobic polymer can include silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, ethylene vinyl acetate copolymers, etc., as well as combinations thereof. The hydrophobic polymer can comprise from about 30 wt. % to about 80 wt. %, such as from about 40 wt. % to about 70 wt. %, such as from about 30 wt. % to about 60 wt. % of the composition.

The hydrophobic polymer can include a biodegradable polymer. Examples of suitable biodegradable polymers for this purpose may include, for instance, polymers made of monomers such as organic esters or ethers, which are capable of degradation in a variety of environments. Anhydrides, amides, ortho esters, by themselves or in combinations with other monomers can also be used. The polymers can be crosslinked or non-crosslinked. The biodegradable polymers can also include various groups such as oxygen-based groups, including oxy, hydroxy, ether, carbonyl, e.g., non-oxo-carbonyl, ester, carboxylic acids, etc., amide cyano, amino, etc. Suitable biodegradable polymers useful for drug delivery are described in Heller, Biodegradable Polymers in Controlled Drug Delivery, In: “CRC Critical Reviews in Therapeutic Drug Carrier Systems”, Vol. 1. CRC Press, Boca Raton, Fla. (1987), which is incorporated by reference herein in its entirety. Specifically, suitable biodegradable polymers include polylactic acid polymers (PLA), poly(D,L) lactide polymers, poly ortho ester (POE) polymers, etc. as well as combinations thereof.

In certain embodiments, the polymer matrix includes a POE polymer. POE polymers contain ortho ester groups, which are functional groups containing three alkoxy groups attached to one carbon atom, along the backbone of the polymer. POE polymers can undergo surface erosion via a hydrolysis mechanism. Suitable POE polymers for use in the present disclosure are further described in U.S. Pat. Nos. 4,093,709A, 4,180,646, and 4,304,767, which are incorporated herein by reference. The POE polymers of the present disclosure can have glass transitions temperatures (T_{g}) ranging from about −20° C. to about 60° C., such as from about −10° C. to about 110° C., such as from about 0° C. to about 100° C., such as from about 10° C. to about 90° C., such as from about 20° C. to about 80° C., such as from about 30° C. to about 70° C., such as from about 40° C. to about 60° C. as determined in accordance with ASTM E1640-18. The POE polymers of the present disclosure can have a weight-average molecular weight ranging from about 1 kDa to 100 kDa, such as from about 5 kDa to about 80 kDa, such as from about 20 kDa to about 50 kDa, such as from about 20 kDa to about 70 kDa.

POE polymers can be synthesized via a variety of processes, including, condensation reactions of furans and dialcohols. POE polymers can also be polymerized via the transesterification of other esters with polyols. Additionally, POE polymers can be polymerized via addition reactions of acetals with various alcohols and/or polyols and, optionally, cyclic esters (e.g., glycolide and/or lactide). In embodiments herein, the POE polymers can include the reaction product formed from an addition reaction with one or more acetal monomers, one or more polyol monomers, and, optionally, one or more cyclic esters. The cyclic esters can be conjugated to the one or more polyol monomers, forming a modified polyol monomer. Such modified polyol monomers can add ester functionality to the POE polymer. As further described below, controlling the nature and amounts of acetals, polyols (including modified polyols), and additional monomers (e.g., cyclic ester monomers), provides for selective control over the total quantity of each monomer in the POE polymer and the total quantity of ester functional groups in the POE polymer. Suitable addition reactions of ketene acetals and polyols are described in U.S. Pat. Nos. 4,304,767, 4,549,010, and 5,968,543, which are incorporated herein by reference. For instance, the POE polymer is produced by copolymerizing an acetal monomer and a polyol monomer in a suitable solvent (e.g., polar solvents). The selected monomers can be dispersed in a polar solvent with trace amounts of acidic catalyst added as needed. The degree of polymerization during the addition reaction can be controlled via adjusting the stoichiometry of the monomers according to the Carothers Equation, shown below.

$$DP = \frac{1 + r}{1 + r - 2 rp}$$

where DP is the average degree of polymerization, r is the excess of one monomer to the other and p is the degree of conversion of the polymerization. Further, in order to control the molecular weight of the POE polymer, one or more monofunctional monomers (e.g., an alcohol instead of a diol) can be added at the beginning of the polymerization. Reaction of the monofunctional alcohol with the ketene acetal monomer blocks the polymerization of the ketene acetal monomer at one end.

In embodiments, the POE polymer can be polymerized by placing one or more acetal monomers and one or more polyol monomers in a suitable solvent in a paddle-stirred flask under anhydrous conditions. Optionally, the anhydrous conditions can be maintained while a catalyst is added. The mixture is stirred for about one hour. The POE polymer can then be isolated from the solution by either precipitation into n-hexane followed by filtration of the white solid or by evaporation of the tetrahydrofuran in Teflon coated pan placed in a vacuum chamber. Suitable solvents include aprotic solvents, such as dimethylacetamide, dimethyl sulfoxide, dimethylformamide, acetonitrile, acetone, ethyl acetate, pyrrolidone, tetrahydrofuran, and methylbutyl ether, and the like. Catalysts are not required for this reaction, but when used, suitable catalysts are iodine in pyridine, p-toluenesulfonic acid; salicylic acid, Lewis acids (such as boron trichloride, boron trifluoride, boron trichloride etherate, boron trifluoride etherate, stannic oxychloride, phosphorous oxychloride, zinc chloride, phosphorus pentachloride, antimony pentafluoride, stannous octoate, stannic chloride, diethyl zinc, and mixtures thereof); and Brønsted catalysts (such as polyphosphoric acid, crosslinked polystyrene sulfonic acid, acidic silica gel, and mixtures thereof). In embodiments, the amount of catalyst used can be about 0.2% by weight relative to the acetal monomer. Smaller or larger amounts can also be used, such as about 0.005% to about 2.0% by weight relative to the acetal monomer.

As noted, acetal monomers can be used to polymerize the POE polymers. As used herein “acetal” refers to compounds including both acetals and ketals. Suitable acetal monomers can include aliphatic acetals, aryl acetals, cyclic acetals, cyclic ketene acetals (CKAs), including bicyclic ketene acetals, and combinations thereof. Suitable ketene acetals are shown below.

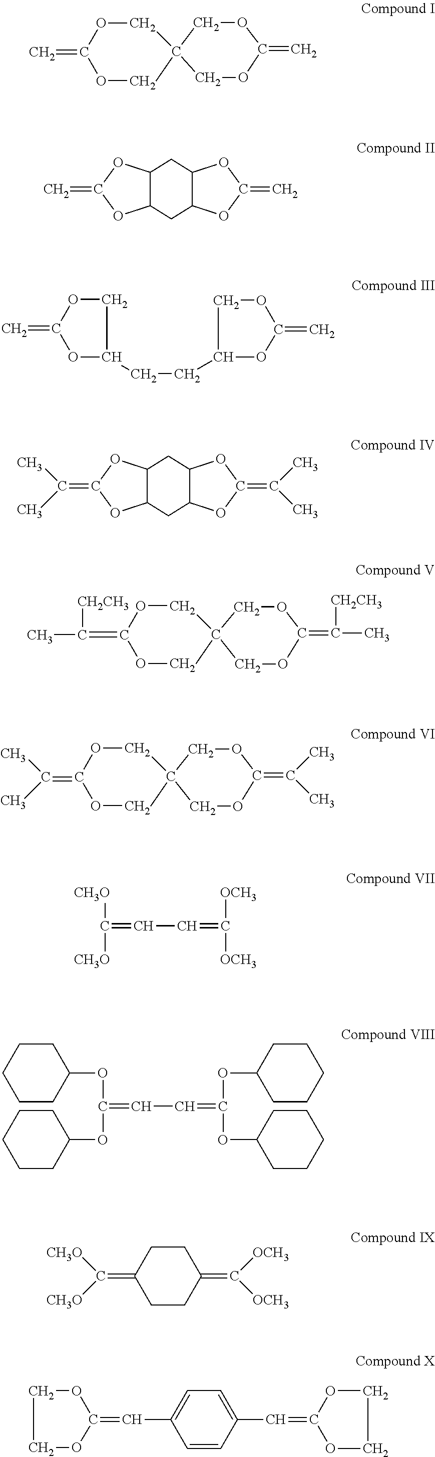

In embodiments, the acetal monomer includes a bicyclic ketene acetal monomer. A suitable bicyclic ketene acetal monomer is 3,9-diethylidene-2,4,8,10-tetraoxaspiro[5.5]undecane (DETOSU), as shown below in Formula 1.

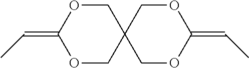

DETOSU has a chemical formula of C₁₁H₁₆O₄ and is derived from the isomeric allyl acetal 3,9-divinyl-2,4,8,10-tetraoxaspior[5.5]undecane (DVTOSU). DETOSU is a bifunctional monomer and is capable of forming POE polymers by the addition of polyols (e.g., diols) to the activated double bond of the diketene acetal. The acetals monomers used herein can include a variety of functionalities including those having a functionality of at least two or greater. It is to be understood that where the acetal monomer has functionalities greater than two, crosslinking of the polymer will likely result. The acetal monomers used herein can include a single acetal monomer or blends and/or combinations of different acetal monomers. The acetal monomer content of the POE polymer may be within a range of from about 20 mol. % to about 90 mol. %, such as from about 30 mol. % to about 80 mol. %, such as from about 40 mol. % to about 60 mol. %. In a specific embodiment, the acetal monomer content of the POE polymer is about 45 mol. % to about 55 mol %, such as about 50 mol. %.

POE polymers of the present disclosure can be synthesized using polyol monomers. As used herein, “polyol” refers to a compound having more than one hydroxyl group. Suitable polyols include triols and the like that can enter into the polymerization reaction without adversely affecting it or the polymeric product. Generally, suitable polyols can include α,ω-aliphatic diols, triols and the like of the straight or branched chain type. Representative polyols are alkane polyols having a terminal hydroxyl group at the terminus of an alkylene chain of the formula:

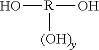

wherein R is an alkylene chain of 2 to 12 carbon atoms and y is 0 to 6. Typical diols include 1,5-pentanediol; 1,6-hexanediol; 1,7-heptanediol; 1,9-nonanediol; 2,3-dimethyl-1,6-hexanediol; 3,6-diethyl-1,9-nonanediol; 1,12-dodecamethanediol; and the like.

Polyols containing more than 2 reactive hydroxyl radicals suitable for use herein include polyhydroxyl compounds such as 1,2,3,4,5,6-hexanehexol; 1,2,3-propanetriol; 1,2,5-pentanetriol; 1,3,5-pentanetriol; 1,2,4-butanetriol; 2-methyl-1,2,3-propanetriol; 2-methyl-2 (hydroxymethyl) 1,2-propanediol; 1,4,7-heptanetriol; 1,5,10-decanetriol; 1,5,12-dodecanetriol; and the like.

Other polyols suitable for synthesizing the polymers include polyglycols containing a repeating glycol monoether moiety —OCH2 (CH₂)ₚOH wherein p is 1 to 5, and the polyglycols are diglycols, triglycols, tetraglycols, and the like. Typical polyglycols include diethylene glycol, triethylene glycol, tetraethylene glycol, bis(4-hydroxybutyl)ether, bis(3-hydroxypropyl)ether, and the like.

Additional polyols that can be used in accordance with the disclosure are polyhydroxyl compounds having 2 or more reactive hydroxyl groups such as pentaerythritol; dipentaerythritol; 3-methylglycerol; 1,4-cyclohexane dicarbinol in the cis, trans isomeric configuration or mixtures thereof; 2,2,4,4-tetramethyl cyclobutane 1,3-diol; adonitol; mannitol; 2,5-dipropyl-1,4-phenyldipropanol; 1,3-cyclopropanol; 2-propenyl-1,4-cyclohexane dipropanol; trimethylol propane; sorbitol; penacol; 2-methyl-1,4-cyclohexane dicarbinol; 3-isopropoxy-1,4-cyclohexane dipropanol; 2-ethenyl-1,3-cyclopentane dicarbinol; 1,4-phenyldicarbinol; 2-propyl-1,4-phenyldiethanol; 3-butoxy-1,4-phenyldibutanol; and the like. The preparation of the above polyols is known to the art and described further in Acta Pharm. Jugaslav. Vol 2 pages 134 to 139, 1952; Ann. Vol. 594, pages 76 to 88, 1955; J. Am. Chem. Soc. Vol 71, pages 3618 to 3621, 1949; ibid., Vol. 74, pages 2674 to 2675, 1952; Chem. Abst., Vol. 42, pages 8774 to 8775, 1948; ibid., Vol 43 pages 571 to 573 and 6652, 1949; ibid., Vol. 44, pages 2554 and 7231, 1950; ibid., Vol. 46, page 9585, 1952; ibid., Vol. 47, page 7575, 1953; ibid., Vol. 48, page 106, 1954, ibid., Vol. 49, pages 6098 to 6099, 1955; Encyclopedia of Chemical Technology, Kirk-Othmer, Vol. 10, pages 638 to 678, 1966, published by Interscience Publishers, New York.

Phenolic polyols (two or more phenolic hydroxyl groups) and mixed phenolic-alcoholic polyols may be employed. Also, mixtures of two or more polyols may be employed. Examples of polyols and of mixed phenolic-alcoholic polyols are as follows: 4,4′-isopropylidenediphenol (bisphenol A); 4-hydroxybenzylalcohol; 4-hydroxy-3-methoxybenzylalcohol; p-hydroxyphenethylalcohol; 4,4′-dihydroxydiphenyl; 4,4′-dihydroxydiphenylmethane; 2,4-dihydroxybenzaldehyde; catechol; resorcinol; hydroquinone; 2,2′-dihydroxybenzophenone; 2,4-dihydroxybenzophenone; and 3,4-dihydroxymethylcinnamate; also non-phenolic polyols having aromatic linking groups between the hydroxyl groups, e.g. 1,4-dihydroxymethylbenzene. Furthermore, tri-(and higher) hydric phenols may be used such as pyrogallol; hydroxyhydroquinone; phloruglucinol; and propyl gallate.

In embodiments, the polyols include hexane-1,6-diol (HDO), cyclohexane dimethanol (CHDM), triethylene glycol (TEG), and combinations thereof. Similar to the acetal monomers, the polyol monomers can have functionalities of at least 2 or greater. It is to be understood that where the polyol monomer has functionalities greater than two, crosslinking of the polymer will likely result. The polyol monomers used herein can include a single polyol monomer or blends and/or combinations of different polyol monomers. The polyol monomer content of the POE polymer may be within a range of from about 20 mol. % to about 90 mol. %, such as from about 30 mol. % to about 80 mol. %, such as from about 40 mol. % to about 60 mol. %. In a specific embodiment, the polyol monomer content of the POE polymer is about 45 mol. % to about 55 mol %, such as about 50 mol. %.

The polyol monomer(s) can be modified to include a polyol having one or more cyclic esters (e.g., glycolide) conjugated thereto. For instance, modified polyols can include prepolymers formed from a reaction between one or more cyclic esters and the polyol (e.g., diol). Thus, the resulting modified polyols can have aliphatic ester units and ester functionality. These modified polyols containing one or more cyclic esters can then be polymerized with one or more acetals to form the POE polymer. For instance, in embodiments, HDO, CHDM, and/or TEG can be reacted with glycolide (GL) forming the following modified polyol monomers CHDM-GL, HDO-GL, and TEG-GL. Resulting modified polyols monomers can then be reacted with acetal monomers to form the POE polymers. Other modified polyols can also be utilized in accordance with the present disclosure. For instance, the polyol monomers can be modified to include different functionalities in order to modify end properties of the POE polymer. In embodiments, from about 0 mol. % to about 100 mol. % of the polyol monomers used to form the POE polymer can include one or more modified polyols, such as from about 0 mol. % to about 75 mol. %, such as from about 10 mol. % to about 60 mol. %, such as from about 20 mol. % to about 50 mol. %. In certain embodiments, the amount of modified polyol can vary based on the type of modified polyol utilized. For instance, in embodiments where CHDM-GL is utilized, the amount of CHDM-GL is greater than about 10 mol. % of the total polyol monomers, such as between about 10 mol. % and about 50 mol. %, such as from about 15 mol. % and about 45 mol. %, such as from about 20 mol. % to about 40 mol. %. In other embodiments, where HDO-GL is utilized, the amount of HDO-GL is less than about 10 mol. % of the total polyol monomers. Indeed, as provided herein, the amount of modified polyol included can vary based on the type of modified polyol selected.

As discovered by the present inventors and further described herein, through control of the type and amount of monomers utilized during polymerization, properties of the POE polymer can be tailored to achieve desired therapeutic agent release from the polymer matrix. For instance, POE polymers can include blocks of acidic monomers (e.g., cyclic esters) distributed throughout the POE polymer backbone. These acidic segments can release acidic byproducts upon hydrolysis of the polymer, which can further catalyze hydrolysis of the POE polymer. Selection of the particular nature and amount of polyol can be used to adjust the glass transition temperature T_{g} and/or the hydrophilicity of the resultant POE polymer. For instance, in order to increase the glass transition T_{g} temperature of the POE polymer higher amounts of CHDM or modified-CHDM (e.g., CHDM-GL) can be utilized. To increase the hydrophilicity of the POE polymer, TEG or modified TEG (e.g., TEG-GL) including higher amounts of TEG can be utilized during polymerization. Further, the type and/or amount of ester functional groups throughout the POE polymer backbone can be modified to tune therapeutic agent release. To increase the ester content of the POE polymer, higher amounts of GL-modified polyols can be incorporated. For instance, HDO-GL can be utilized to increase the overall ester content of the POE polymer. Thus, selection of the particular monomers and amounts of the monomers can be used to adjust mechanical and thermal properties of the POE polymer.

In certain embodiments, it may also be desirable to employ blends of POE polymers having different mechanical and thermal properties. For instance, the polymer matrix can include a first POE polymer and a second POE polymer such that the overall blend and polymer matrix have a T_{g}, and/or molecular weight within the range noted above. For example, the polymer matrix may contain a first biodegradable polymer (e.g., POE polymer) and a second biodegradable polymer (e.g., POE polymer) having a T_{g} that is greater than the T_{g} of the first polymer. The second polymer may likewise have a molecular weight that is the same, lower, or higher than the corresponding molecular weight of the first polymer. For instance, the first POE polymer can have a T_{g} that is from about −10° C. to about 50° C., such as from about 0° C. to about 30° C., such as from about 10° C. to about 30° C. The first POE polymer can have a T_{g} that is greater than 0° C. The second POE polymer can have a T_{g} ranging from 0° C. to about 50° C., such as from about 10° C. to about 40° C., such as from about 15° C. to about 35° C. In such embodiments, the second biodegradable polymer can also include a molecular weight that is greater than the molecular weight of the first biodegradable polymer. The first polymer may constitute from about 20 wt. % to about 80 wt. %, in some embodiments from about 30 wt. % to about 70 wt. %, and in some embodiments, from about 40 wt. % to about 60 wt. % of the injectable composition, and the second polymer may likewise constitute from about 20 wt. % to about 80 wt. %, in some embodiments from about 30 wt. % to about 70 wt. %, and in some embodiments, from about 40 wt. % to about 60 wt. % of the injectable composition.

Further, in embodiments, the amount of ester functionality in the polymer component of the injectable composition can be controlled by blending a first POE polymer having a high ester content with a second POE polymer having a lower ester content as compared to the first POE polymer. Control of the amount of ester functionality in the resultant POE polymer mixture can affect the hydrolyzation and degradation of the POE polymer. For instance, ester linkages in the backbone of the POE polymer are more easily hydrolyzed as compared to other functional groups. Hydrolysis of the ester group produces acid that catalyzes further decomposition of the POE polymer. Thus, POE polymers having greater amounts of ester in the polymer or polymer blend will degrade faster that those containing lesser amounts of esters.

Typically, biodegradable polymer(s), such as POE polymers, constitute the entire content of the hydrophobic polymers present in the composition. In other words, the composition may be substantially free of non-biodegradable, hydrophobic polymers, such as silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, ethylene vinyl acetate copolymers, etc., as well as combinations thereof. If used, additional hydrophobic polymers (e.g., those used in addition to POE polymers) generally constitute no more than 10 wt. % of the composition, in some embodiments no more than about 5 wt. % of the composition, and in some embodiments, from 0 wt. % to about 2 wt. % of the composition (e.g., 0 wt. %).

In certain embodiments, the composition can further include one or more hydrophilic polymers, such as celluloses, including ethylcellulose, methylcellulose, hydroxymethylcellulose, etc., polyvinylpyrrolidone, and so forth. In such embodiments, the POE polymers may be blended with other types of hydrophilic polymers. In such embodiments, POE polymers may constitute from about from about 70 wt. % to about 99.999 wt. %, in some embodiments from about 80 wt. % to about 99.99 wt. %, and in some embodiments, from about 90 wt. % to about 99.9 wt. % of the polymer content of the composition, while other hydrophilic polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the composition.

### B. Solvents

The injectable flowable gel composition(s) as disclosed include one or more solvents. For instance, the hydrophobic polymer (e.g., POE polymer) is dissolved within a solvent to render the flowable composition. The solvent can have a miscibility in water that is suitable for dissolving the hydrophobic polymer and forming a flowable viscous gel. For instance, in embodiments, the composition comprises from about 50 mg to 4,000 mg of POE polymer per milliliter of solvent, such as from about 350 mg to about 3000 mg of POE polymer per milliliter of solvent, such as from about 450 mg to about 4000 mg of POE polymer per milliliter of solvent, such as from about 340 mg of POE polymer to about 1,000 mg of POE polymer per milliliter of solvent. In embodiments, the concentration of the POE polymer(s) in the solvent can be from about 5% w/w to about 400% w/w, such as from about 20% w/w to about 350% w/w, such as from about 50% w/w to about 300% w/w, such as from about 100% w/w to about 200% w/w. As further described hereinbelow, the amount of POE polymer and therapeutic agent can be measured and then dissolved in the desired amount of solvent as disclosed herein to form an injectable composition.

Any suitable biocompatible solvent can be utilized in accordance with the present disclosure. In embodiments, the solvent includes an organic solvent. Suitable organic solvents include N-methyl-2-pyrrolidone (NMP), 2-pyrrolidone, dimethyl sulfoxide (DMSO), benzyl benzoate, benzyl alcohol, triacetin, glycofurol, polyethylene glycol, ethyl acetate, and combinations and/or mixtures thereof. In embodiments, the solvent includes aromatic alcohols; lower alkyl and aralkyl esters of aryl acids; aryl, aralkyl and lower alkyl ketones; lower alkyl esters of citric acid; and mixtures thereof. Other suitable organic solvents can include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diethylene glycol dimethyl ether, 1,2-dimethoxy-ethane, dimethyl-formamide, 1,4-dioxane, ethanol, ethylene glycol, glycerin, heptane, hexamethylphosphoramide, hexamethylphosphorous triamide, hexane, methanol, methyl t-butyl ether, methylene chloride, nitromethane, pentane, petroleum ether, 1-propanol, 2-propanol, pyridine, tetrahydrofuran (THF), toluene, triethyl amine, water, heavy water, o-xylene, m-xylene, p-xylene, and combinations thereof.

As provided herein, through selective control over the type and amount of solvent, release properties for the injectable flowable composition can be modified and/or controlled. For instance, upon injection, the solvent(s) disperses from the implant into the extracellular environment, thus forming a in situ implant from the POE polymer and therapeutic agent. Selection of the particular solvent can affect diffusion of the solvent from the composition, which, in turn can affect release of the therapeutic agent from the composition. For instance, solvents having a higher solubility in water (e.g., NMP), will more readily diffuse from the implant, which can facilitate faster or burst release of the therapeutic agent. Utilization of other solvents (e.g., ethyl acetate) having lower solubility in water, will diffuse more slowly, which, in turn, can decrease the initial release or burst release of the therapeutic agent. Thus, in embodiments, the solvent can include combinations and mixtures of solvents having different solubilities in water in order to tune release of the therapeutic agent.

### C. Therapeutic Agents

One or more therapeutic agents are also provided in the composition that are capable of prohibiting and/or treating a condition, disease, and/or cosmetic state of a patient. Therapeutic agents are distinguished from other components such as matrices, carriers, diluents, excipients, additives, etc. or other protective components. The therapeutic agent may be prophylactically, therapeutically, and/or cosmetically active, systemically or locally. Suitable therapeutic agents will be further discussed hereinbelow.

In embodiments, the therapeutic agent includes one or more basic therapeutic agents. As used herein, the term “basic therapeutic agent” means a therapeutic agent that is an acceptor of a proton and has a pKa of 4 and above.

General examples of therapeutic agents include, but are not limited to, anti-infectives (including antibiotics, antivirals, fungicides, scabicides or pediculicides); antiseptics (e.g., benzalkonium chloride, benzethonium chloride, chlorhexidine gluconate, mafenide acetate, methylbenzethonium chloride, nitrofurazone, nitromersol and the like); steroids (e.g., estrogens, progestins, androgens, adrenocorticoids, and the like); antiallergenics (e.g., sodium chromoglycate, antazoline, methapyriline, chlorpheniramine, cetrizine, pyrilamine, prophenpyridamine); miotics and anti-cholinesterase (e.g., pilocarpine, salicylate, carbachol, acetylcholine chloride, physostigmine, eserine, diisopropyl fluorophosphate, phospholine iodine, demecarium bromide); hormonal agents (e.g., estrogens, estradiol, progestational, progesterone, insulin, calcitonin, parathyroid hormone, peptide and vasopressin hypothalamus releasing factor); analgesics and anti-inflammatory agents (e.g., aspirin, ibuprofen, naproxen, ketorolac, COX-1 inhibitors, COX-2 inhibitors, salicylate, indomethacin, diclofenac, flurbiprofen, piroxicam, other non-steroidal anti-inflammatoires, and the like); narcotics (e.g., morphine, meperidine, codeine, and the like); local anesthetics (e.g., the amide- or anilide-type local anesthetics such as bupivacaine, dibucaine, mepivacaine, procaine, lidocaine, tetracaine, and the like); anti-proliferative agents (e.g., 1,3-cis retinoic acid); decongestants (e.g., phenylephrine, naphazoline, tetrahydrazoline); antineoplastics (e.g., carmustine, cisplatin, fluorouracil); immunological drugs (e.g., vaccines and immune stimulants); chemotherapeutic agents (e.g., mechlorethamine, cyclophosphamide, fluorouracil, thioguanine, carmustine, lomustine, melphalan, chlorambucil; streptozocin, methotrexate, vincristine, bleomycin, vinblastine, vindesine, dactinomycin, daunorubicin, doxorubicin, tamoxifen, and the like); immunosuppressive agents, growth hormone antagonists, growth factors (e.g., epidermal growth factor, fibroblast growth factor, platelet derived growth factor, transforming growth factor beta, somatotropin, fibronectin); inhibitors of angiogenesis (e.g., angiostatin, anecortave acetate, thrombospondin, etc.); interferons (e.g., interferon alpha-2b, peg interferon alpha-2a, interferon alpha-2b+ribavirin, pegylated interferon-2a, interferon beta-1a, interferon beta); interleukins (e.g., interleukin-2); vaccines (e.g., whole viral particles, recombinant proteins, subunit proteins, gp41, gp120, gp140, DNA vaccines, plasmids, bacterial vaccines, polysaccharides, extracellular capsular polysaccharides); dopamine agonists; radiotherapeutic agents; therapeutic polypeptides and/or proteins (e.g., insulin, erythropoietin, morphogenic proteins such as bone morphogenic protein, and the like); enzymes; extracellular matrix components; ACE inhibitors; free radical scavengers; chelators; antioxidants; anti-polymerases; photodynamic therapy agents; gene therapy agents; corticosteroids (e.g., dexamethasone), tyrosine kinase inhibitors (e.g., axitinib, bosutinib, cabozantinib, crizotinib, dasatinib, erlotinib, gefitinib, imatinib, lapatinib, nilotinib, pazopanib, ponatinib, ruxolitinib, sorafenib, sunitinib, vatalanib, vemurafenib, etc.), and so forth, as well as combinations of any of the foregoing.

In certain embodiments, the therapeutic agent may be a macromolecular compound having a relatively large molecular weight, such as about 1 kilodaltons (kDa) or more, in some embodiments from about 2 kDa to about 1000 kDa, in some embodiments from about 20 kDa to about 950 kDa, in some embodiments from about 50 kDa to about 750 kDa, and in some embodiments, from about 100 kDa to about 500 kDa, or any range therebetween. The macromolecular compound may, for instance, include a protein, peptide, enzyme, antibody, interferon, interleukin, blood factor, vaccine, nucleotide, lipid, or an analogue, derivative, or combination thereof. Alternatively, small molecule therapeutic agents may also be employed, such as those having a molecular weight of less than about 1,000 Da, in some embodiments about 900 Da or less, in some embodiments from about 10 to about 800 Da, and in some embodiments, from about 20 to about 700 Da, or any range therebetween. The weight ratio of the therapeutic agent to the POE polymer in the composition can range from about 0.01 to about 5, such as from about 0.1 to about 4.7, such as from about 0.5 to about 4.5, such as from about 1 to about 4, such as from about 2 to about 3, such as from about 0.5 to about 1.75, such as from about 0.7 to about 1.5, such as from about 1 to about 1.2. The therapeutic agent(s) can comprise from about 5 wt. % to about 95 wt. % of the composition, such as from about 10 wt. % to about 90 wt. %, such as from about 15 wt. % to about 85 wt. %, such as from about 20 wt. % to about 80 wt. %, such as from about 25 wt. % to about 75 wt. %, such as from about 30 wt. % to about 70 wt. %, such as from about 35 wt. % to about 65 wt. %, such as from about 40 wt. % to about 60 wt. %, such as from about 45 wt. % to about 55 wt. %, such as from about 40 wt. % to about 50 wt. %.

If desired, in some embodiments, the composition may be suited to deliver a nucleic acid as a therapeutic agent. The therapeutic agent can include one or more nucleic acids. As used herein, the term “nucleic acid” generally refers to a compound comprising a nucleobase and an acidic moiety, e.g., a nucleoside, nucleotide, polynucleotide, or a combination thereof. A “nucleoside” generally refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” generally refers to a nucleoside including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides. For example, polynucleotides may contain three or more nucleotides in which adjacent nucleotides are linked to each other via a phosphodiester linkage. The term “nucleic acid” also encompasses RNA as well as single and/or double-stranded DNA. More particularly nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-c-LNA having a 2′-amino functionalization), ethylene nucleic acids (ENA), cyclohexenyl nucleic acids (CeNA) or chimeras or combinations thereof.

Nucleic acids may be naturally occurring, for example, in the context of a genome, a transcript, a mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Nucleic acids can be purified from natural sources, produced using recombinant expression systems and optionally purified, chemically synthesized, etc. The nucleic acids may also include nucleoside analogs, such as analogs having chemically modified bases or sugars, and backbone modifications. In some embodiments, the nucleic acid is or contains natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Modified nucleotide base pairing may be employed and encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In certain embodiments, the nucleic acid may be a polynucleotide (e.g., RNA polynucleotides, such as mRNA polynucleotides) in which one or more nucleobases has been modified for therapeutic purposes. In fact, in certain embodiments, a polynucleotide (e.g., RNA polynucleotide, such as mRNA polynucleotide) may be employed that includes a combination of at least two (e.g., 2, 3, 4 or more) modified nucleobases. For example, suitable modified nucleobases in the polynucleotide may be a modified cytosine, such as 5-methylcytosine, 5-methyl-cytidine (m5C), N4-acetyl-cytidine (ac4C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine, etc.; modified uridine, such as 5-cyano uridine, 4′-thio uridine, pseudouridine (ψ), N1-methylpseudouridine (m1ψ), N1-ethylpseudouridine, 2-thiouridine (s2U), 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine (mo5U), 5-methoxyuridine, 2′-O-methyl uridine, etc.; modified guanosine, such as α-thio-guanosine, inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine, etc.; modified adenine, such as α-thio-adenosine, 7-deaza-adenine, 1-methyl-adenosine (m1A), 2-methyl-adenine (m2A), N6-methyl-adenosine (m6A), 2,6-diaminopurine, etc.; as well as combinations thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the polynucleotide (e.g., RNA polynucleotide, such as mRNA polynucleotide) may be uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, polynucleotides function as messenger RNA (mRNA). “Messenger RNA” (mRNA) generally refers to any polynucleotide that encodes a (at least one) polypeptide (a naturally occurring, non-naturally occurring, or modified polymer of amino acids) and can be translated to produce the encoded polypeptide in vitro, in vivo, in situ or ex vivo. The basic components of a mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features that serve to overcome existing problems of effective polypeptide expression using nucleic-acid based therapeutics. The mRNA may contain at least one (one or more) ribonucleic acid (RNA) polynucleotide having an open reading frame encoding at least one polypeptide of interest. In some embodiments, a RNA polynucleotide of a mRNA encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, a RNA polynucleotide of a mRNA encodes at least 10, 20, 30, 40, 50, 60, 70, 80, 90 or 100 polypeptides. In some embodiments, an RNA polynucleotide of a mRNA encodes at least 100 or at least 200 polypeptides.

In some embodiments, the nucleic acids are therapeutic mRNAs. As used herein, the term “therapeutic mRNA” refers to a mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of various diseases and conditions, such as bacterial infections, viral infections, parasitic infections, cell proliferation disorders, genetic disorders, and autoimmune disorders. The mRNA may be designed to encode polypeptides of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery.

Particularly suitable therapeutic mRNAs are those that include at least one ribonucleic acid (RNA) polynucleotide having an open reading frame encoding at least one antigenic polypeptide, in which the RNA polynucleotide of the RNA includes at least one chemical modification. The chemical modification may, for instance, be pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine, and 2′-O-methyl uridine.

Although by no means required, the particular nature of the nucleic acid may also be selected to help improve its ability to be dispersed within the polymer matrix and delivered to a patient without significant degradation. For instance, it may be desired to co-deliver a conventional RNA (e.g., mRNA) with a self-amplifying RNA. Conventional mRNAs, for instance, generally include an open reading frame for the target antigen, flanked by untranslated regions and with a terminal poly(A) tail. After transfection, they drive transient antigen expression. Self-amplifying mRNAs, on the other hand, are capable of directing their self-replication, through synthesis of the RNA-dependent RNA polymerase complex, generating multiple copies of the antigen-encoding mRNA, and express high levels of the heterologous gene when they are introduced into the cytoplasm of host cells. Circular RNA (circRNA), which is a single-stranded RNA joined head to tail, may also be employed. The target RNA may be circularized, for example, by backsplicing of a non-mammalian exogenous intron or splint ligation of the 5′ and 3′ ends of a linear RNA. Examples of suitable circRNAs are described, for instance, in U.S. Patent Publication No. 2019/0345503, which is incorporated herein by reference thereto. Antisense RNA may also be employed, which generally has a base carried on a backbone subunit composed of morpholino backbone groups and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. Morpholino oligonucleotides with uncharged backbone linkages, including antisense oligonucleotides, are detailed, for example, in U.S. Pat. Nos. 5,698,685, 5,217,866, 5,142,047, 5,034,506, 5,166,315, 5,185, 444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

In certain cases, the nucleic acid may be an aptamer, such as an RNA aptamer. An RNA aptamer may be any suitable RNA molecule that can be used on its own as a stand-alone molecule, or may be integrated as part of a larger RNA molecule having multiple functions, such as an RNA interference molecule. For example, an RNA aptamer may be located in an exposed region of a shRNA molecule (e.g., the loop region of the shRNA molecule) to allow the shRNA or miRNA molecule to bind a surface receptor on the target cell. After it is internalized, it may then be processed by the RNA interference pathways of the target cell. The nucleic acid that forms the nucleic acid aptamer may include naturally occurring nucleosides, modified nucleosides, naturally occurring nucleosides with hydrocarbon linkers (e.g., an alkylene), and/or or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid aptamer can be replaced with a hydrocarbon linker or a polyether linker. Suitable aptamers are described, for instance, in U.S. Pat. No. 9,464,293, which is incorporated herein by reference thereto.

Protein-fused nucleic acids may also be suitable for use in the present invention. For example, proteins (e.g., antibodies) may be covalently linked to RNA (e.g., mRNA). Such RNA-protein fusions may be synthesized by in vitro or in situ translation of mRNA pools containing a peptide acceptor attached to their 3′ ends. In one embodiment, after readthrough of the open reading frame of the message, the ribosome pauses when it reaches the designed pause site, and the acceptor moiety occupies the ribosomal A site and accepts the nascent peptide chain from the peptidyl-tRNA in the P site to generate the RNA-protein fusion. The covalent link between the protein and the RNA (in the form of an amide bond between the 3′ end of the mRNA and the C-terminus of the protein that it encodes) allows the genetic information in the protein to be recovered and amplified (e.g., by PCR) following selection by reverse transcription of the RNA. Once the fusion is generated, selection or enrichment is carried out based on the properties of the mRNA-protein fusion, or, alternatively, reverse transcription may be carried out using the mRNA template while it is attached to the protein to avoid the impact of the single-stranded RNA on the selection. Examples of such protein-fused nucleic acids are described, for instance, in U.S. Pat. No. 6,518,018, which is incorporated herein by reference. Ribozymes (e.g., DNAzyme and/or RNAzyme) may also be employed that are conjugated to nucleic acids having a sequence that catalytically cleaves RNA, such as described in U.S. Pat. No. 10,155,946, which is incorporated herein by reference.

Apart from single strand nucleic acids such as described above, various specific types of double strand nucleic acids may also be employed to help improve stability. Circular DNA (cDNA) and plasmid nucleic acids (e.g., pDNA), which are a closed circular form of DNA, may be employed in certain embodiments. Examples of such nucleic acids are described, for instance, in WO 2004/060277 which is incorporated herein by reference. Long double stranded DNA may also be employed. For instance, a scaffolded DNA origami may be employed in which the long single-stranded DNA is folded into a certain shape by annealing the scaffold in the presence of shorter oligonucleotides (“staples”) containing segments or regions of complementary sequences to the scaffold. Examples of such structures are described, for instance, in U.S. Patent Publication Nos. 2019/0142882 and 2018/0171386, which are incorporated herein by reference.

Antisense RNA (e.g., ASOs) may also be employed, which generally has a base carried on a backbone subunit, containing one or more backbone groups (e.g., phosphorothioates or morpholino backbone groups) and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. ASOs are complementary to a section of naturally occurring RNA, such as mRNA or viral RNA, to form Watson-Crick base pairs and thus to inhibit a biological function of the RNA. As used herein “antisense oligonucleotide” refers to a nucleotide sequence which is substantially complementary to a target nucleotide sequence in a pre-mRNA molecule, hrRNA (heterogenous nuclear RNA), or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions.

The length of the ASO can range from about 5 nucleotide or nucleoside subunits in length to about 50 nucleotide or nucleoside subunits in length. As will be appreciated, a subunit is a nucleobase and a sugar combination (e.g., ribose or deoxyribose) suitably bound to adjacent subunits through phosphorus or non-phosphorus linkages, as further discussed hereinbelow. The length of the ASO can range from about 10 subunits to about 45 subunits, such as from about 15 subunits to about 40 subunits, such as from about 20 subunits to about 35 subunits. In certain embodiments, the ASO has a length of from about 10 subunits to about 25 subunits. The length of the ASO can vary so long as it is capable of binding selectively to the intended location on an RNA target.

Natural, or unmodified, oligonucleotides are susceptible to nuclease degradation and poor protein binding. Accordingly, unmodified oligonucleotides have inefficient tissue uptake precluding their use as clinically effective therapeutic agents. Modifications to the oligonucleotides, however, have been shown to decrease nuclease degradation. For example, multiple types of modifications to internucleoside linkages in nucleotides or nucleosides can improve various properties of the ASO. Chemical modifications to the phosphodiester backbone can be made in order to improve pharmacokinetic properties, tolerability profile, and/or target binding affinity. For instance, certain modifications to the phosphodiester backbone that can be utilized in ASOs of the present disclosure include the following: phosphorothioate ODN (PS-ODN), R-isomer of PS-ODN, (Rp-PS-ODN), S-isomer of PS-ODN, (Sp-PS-ODN), methylphosphonate ODN (PM-ODN), phosphoramidate ODN (PN-ODN) where R═H or alkyl, phosphomopholidate, phosphopiperazidate, phosphorodiamidate morpholino (PMO), and peptide nucleic acids (PNA). ASOs of the present disclosure can also include tricyclo-DNAs (tcDNA) subunits as part of the oligomer. The ASO can include a nucleoside having one or more chemically modified internucleoside linkages. As used herein “chemically modified” when referring to internucleoside linkages includes any of the chemical modifications provided hereinabove with respect to (b)-(j). In certain embodiments, at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 100%, of the internucleoside linkages in the ASO are chemically modified. In other embodiments, chemically modified internucleoside linkages can be disposed about the 3′ end or the 5′ end of the ASO. For instance, at least one and up to five of the internucleoside linkages extending from either the 3′ terminus or the 5′ terminus can include chemically modified internucleoside linkages. In certain embodiments, from about 50% to about 100% of the internucleoside linkages are phosphorothioate linkages (shown in (b), (c), and (d) above). In other embodiments, from about 50% to about 100% of the internucleoside linkages are morpholino subunits (shown in (i) above). Morpholino oligonucleotides with uncharged backbone linkages are detailed, for example, in U.S. Pat. Nos. 5,698,685, 5,217,866, 5,142,047, 5,034,506, 5,166,315, 5,185, 444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

Modifications to the 2′ position of the sugar moiety of the nucleoside subunit is also contemplated. For example, the 2′-position on the sugar moiety can be substituted with a 2′-substituent group. Such 2′-substituent groups can include 2′-fluoro, 2′-alkoxy, 2′-aminoalkoxy, 2′-allyloxy, 2′-imidazole-alkoxy and 2′-poly(ethylene oxide). Alkoxy and aminoalkoxy groups generally include lower alkyl groups, particularly C1-C9 alkyl. Poly(ethylene glycols) are of the structure (O—CH2-CH2)n-O-alkyl. Other 2′-substituent groups can include 2′-O-methyl (2′-OMe), 2′-O-methoxyethyl (2′-MOE) and locked nucleic acid (LNA). The use of 2′-substituent groups can increase the binding affinity of the substituted oligonucleotides or can prevent nuclease degradation in vivo. Notably, at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 100% of nucleotides in the ASO can include modified 2′-substituent groups.

The ASOs of the present disclosure can also include modified bases in the nucleotide units that make up the oligonucleotides of the ASO. Such modified bases may include 6-azapyrimidines and N-2, N-6 and O-6 substituted purines including 2-aminopropyladenine. Other modified pyrimidine and purine base are expected to increase the binding affinity of oligonucleotides to a complementary strand of nucleic acid.

Notably, the ASOs of the present disclosure can include “first generation” ASOs, which refer to oligodeoxynucleotides having 2′-deoxy ribonucleotides and phosphorothioate internucleoside linkages. Suitable ASOs can also include ASOs that are oligonucleotides having a 2′-deoxy “gap” region flanked by “wings” having nucleotides with 2′-modified ribonucleotides, referred to as “gapmers”. Suitable second generation ASOs include an “MOE gapmer” in which the 2′-modified ribonucleotide is a 2′-O-methoxyethyl (2′-MOE or simply MOE) modification, and each of the internucleoside linkages is a phosphorothioate. Second generation ASOs may have a length of 20 nucleotides of which the 5 nucleotides at each terminus are 2′-MOE nucleotides and the center ten nucleotides are 2′-deoxyribonucleotides. Accordingly, these second generation ASOs are referred to as “5-10-5 MOE gapmers” since they have a 5-10-5 wing-gap-wing motif. Suitable 5-10-5 gapmers can have the following formula:

5′mB-mB-mB-mB-mB-dB-dB-dB-dB-dB-dB-dB-dB-dB-dB-mB-mB-mB-mB-mB 3′

where mB=2′-O-methoxyethyl ribonucleoside; dB=deoxynucleoside; “-”=phosphorothiate linkage, and b=heterocyclic base.

The ASO can include a gap region having at least 5 to about 15, such as about 8 to about 10, contiguous 2′-deoxyribonucleosides and a first wing region and a second wing region flaking the gap region. Each of the first and second wing regions independently can include 1 to about 8 2′-O-(2-methoxyethyl) ribonucleotides. The ASOs of the present disclosure can also include “hemimers,” which are chimeric compounds in which there is a single 2′-modified “wing” adjacent to (on either the 5′, or the 3′ side of) a 2′-deoxy gap. The wing present in the hemimer can include 2 nucleotides to about 8 nucleotides. Notably, the gap region, first wing region, or second wing region, can include any of the internucleoside chemical modifications or 2′-substituent groups as disclosed herein.

The ASOs of the present disclosure can also include ASO conjugates, where one or more moieties are conjugated to the ASO. For instance, attached moieties can include peptides, proteins, carbohydrates, fatty acids, aptamers, and small molecules including cholesterol, tocopherol, and folic acid. Specifically, the ASO can also be conjugated to anisamide, stearic acid, cyclic RGD peptide, anandamide, N-acetylgalactosamine, spermine, and combinations thereof. For ASO conjugates, the selected moiety can be conjugated to the ASO in a variety of locations, however, in certain embodiments, the selected moiety is tethered to the 3′-terminus or the 5′-terminus. Different linkers can be used to conjugate the moiety to the ASO. Suitable linkers can include thiol linkers, amino linkers, aldehyde linkers, azide linkers, carboxyl linkers, dibenzylcyclooctyne linkers. ASOs (e.g., morpholino oligonucleotides) can be conjugated to arginine-rich cell penetrating peptides (CPPs). ASOs can also be conjugated to polyethylene glycol (PEG) moieties. The ASO conjugates can include saccharides, such as the monosaccharide GalNAc. For instance, up to three GalNac molecules can be attached to the ASO via a tridentate linker to the 3′-terminus of the ASO. These moieties can be attached to the ASO to improve uptake mechanisms and pharmacokinetic properties of the ASO. In certain embodiments, the ASO can include a dynamic polyconjugate, which is a macromolecule having an ASO that undergoes structural modification in vivo to yield the ASO. Suitable ASO conjugates are further described in U.S. 2016/0289677, WO 2004/044141, WO 2009/073809, WO 2012/083046, WO 2012/089352, WO 2012/089602, WO 2005/086775, WO 2011/126937, WO 2009/025669, which are incorporated by reference herein.

In certain embodiments, the ASO can include an oligonucleotide that has been approved by the U.S. Food and Drug Administration (FDA). Certain approved oligonucleotides include Vitravene™ (Formiversen), Macugen™ (Pegaptanib), Kynamro™ (Mipomersen), Defitelio™ (Defibrotide), Exondys 51™ (Eteplirsen), Spinraza™ (Nusinersen), Tegsedi™ (Inotersen), Onpattro™ (Patisiran), Waylivra™ (Volanesoren), Givlaari™ (Givosiran), Vyondys 53 (Golodirsen), Viltepso™ (Vitolarsen), Oxlumo™ (Lumasiran), Leqvio™ (Inclisiran), Amondys 45™ (Casimersen), and combinations thereof. Other ASOs can include Camlingo™ (Alicaforsen), Drisapersen (PR0051, GSK-2402968), Custirsen (OGX-011 and CC-8490), Miraversen (SPC3649), ISIS-TTR02, Aezea™ (Cenersen), GTI-2040, Imetelstat (GRN163L), ISIS-STAT3RX, Liposomal Grb-2 (BP-100-1.01), and combinations thereof.

In embodiments, the nucleic acid can include an encapsulated nucleic acid that is encapsulated or coated via a carrier component. The molar ratio of the carrier component to the nucleic acid (e.g., mRNA) in the particles may vary, but is typically from about 2:1 to about 50:1, in some embodiments from about 5:1 to about 40:1, in some embodiments from about 10:1 to about 35:1, and in some embodiments, from about 15:1 to about 30:1.

As noted above, the carrier component includes a carrier, such as peptides (e.g., RALA), proteins, carbohydrates (e.g., sugars), polymers (e.g., dextran polymers, such as diethylaminoethyl-dextran; polyethyleneimine; poly(amino ester), aliphatic polyesters, such as polylactic acid, etc.), lipids, and so forth, as well as combinations of any of the foregoing, such as peptide/polymer hybrids (e.g., RALA-PLA). In one particular embodiment, for example, the carrier component may be a lipid component that includes one or more lipids. The nature of such lipid particles may generally vary as is known to those skilled in the art. In one embodiment, for example, the lipid component is a lipid vesicle (e.g., liposome) that includes one or more types and/or layers of lipids. For example, liposomes generally include a phospholipid that is capable of assembling into one or more lipid bilayers. The phospholipid has a phospholipid moiety and optionally one or more additional moieties (e.g., fatty acid moiety). The phospholipid moiety may include, for instance, phosphatidyl choline, phosphatidyl ethanolamine, phosphatidyl glycerol, phosphatidyl serine, phosphatidic acid, 2-lysophosphatidyl choline, and a sphingomyelin. When employed, the fatty acid moiety may likewise include, for instance, lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, docosahexaenoic acid, etc. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition. Examples of suitable phospholipids may include, for instance, alkyl phosphocholines, such as hexadecyl thiophosphocholine, tetradecyl phosphocholine, hexadecyl phosphocholine, docosanoyl phosphocholine, 1,2-dihexadecyl-rac-glycero-3-phosphocholine, DL-α-lysophosphatidylcholine-r-o-hexadecyl, etc.; fatty acid-modified phosphocholines, such as 1,2-distearoyl-sn-glycero-3-phosphocholine (DSPC), 1,2-dioleoyl-sn-glycero-3-phosphoethanolamine (DOPE), 1,2-dilinolenoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, etc.; as well as mixtures of any of the foregoing.

If desired, the lipid component of the vesicles may also include other types of lipids. For example, the lipid component may contain one or more structural lipids to help mitigate aggregation of other lipids in the particles. Examples of suitable structural lipids may include, for instance, steroids; sterols, such as cholesterol, fecosterol, sitosterol, ergosterol, campesterol, stigmasterol, brassicasterol, phytosterols, etc.; glycoalkaloids, such as tomatidine, tomatine, etc.; terpenoids, such as ursolic acid; tocopherols, such as alpha-tocopherol; hopanoids, stanol esters; as well as mixtures thereof. The lipid component of may also include one or more PEG-conjugated lipids to help improve the colloidal stability of the particles in biological environments by reducing a specific absorption of plasma proteins and forming a hydration layer over the particles. Examples of suitable PEG-conjugated lipids may include, for instance, PEG-modified phosphatidylethanolamines, PEG-modified phosphatidic acids, PEG-modified ceramides, PEG-modified dialkylamines, PEG-modified diacylglycerols, PEG-modified dialkylglycerols, etc., as well as mixtures thereof. For example, a PEG lipid may be (R-3-[(Ω-methoxy-poly(ethyleneglycol) 2000) carbamoyl)]-1,2-dimyristyloxypropyl-3-amine) (PEG-c-DOMG), PEG-distearoyl glycerol (PEG-DMG), PEG-1,2-dipalmitoyl-sn-glycero-3-phosphocholine (PEG-DPPC), PEG-DLPE, PEG-DMPE, PEG-DSPE, etc., as well as mixtures thereof. The PEG-conjugated lipid may also be modified to include a hydroxyl group on the PEG chain to form a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid includes an —OH group at the terminus of the PEG chain.

Besides lipid vesicles, other types of lipid particles may also be employed to encapsulate the nucleic acid. Solid lipid particles, for instance, may be employed in certain embodiments of the present invention. Generally speaking, such solid particles are in the form of nanoparticles having a mean diameter of from about 10 to about 1,000 nanometers, in some embodiments from about 20 to about 800 nanometers, in some embodiments from about 30 to about 600 nanometers, and in some embodiments, from about 40 to about 300 nanometers, such as determined by laser diffraction techniques. Similar to lipid vesicles, solid particles also contain a lipid component that includes one or more types and/or layers of lipids.

In one embodiment, for example, the lipid component of the solid particles includes a cationic lipid. Besides a cationic lipid, the lipid component of the solid particles may also include other types of lipids. For example, the lipid component may contain a helper lipid, which is generally neutral or non-cationic at physiological pH. Such helper lipids may include phospholipids such as described above, fatty acids, glycerolipids (e.g., mono-, di-, and triglyceride), prenol lipids, and so forth. Suitable fatty acids may include those having a fatty acid of at least 8 carbon atoms, such as unsaturated fatty acids (e.g., myristoleic acid, palmitoleic acid sapienic acid, oleic acid, elaidic acid, vaccenic acid, linoleic acid, linoelaidic acid, alpha-linoelaidic acid arachidonic acid, eicosapentaenoic acid, erucic acid, docosahexanoic acid, etc., or any cis/trans double-bond isomers thereof), saturated fatty acids (e.g., caprylic acid, capric acid, lauric acid, myristic acid, palmitic acid, stearic acid, arachidic acid, behenic acid, lignoceric acid, cerotic acid, etc., or any cis/trans double-bond isomers thereof), as well as combinations thereof. Particularly suitable helper lipids may include, for instance, oleic acid or an analog thereof, as well as fatty acid-modified phospholipids, such as 1,2-distearoyl-sn-glycero-3-phosphocholine (DSPC) and/or 1,2-dioleoyl-sn-glycero-3-phosphoethanolamine (DOPE), as well as analogs of such compounds in which the phosphocholine moiety is replaced by a different zwitterionic group, such as an amino acid or a derivative thereof.

The formation of solid lipid particles that encapsulate a nucleic acid may be accomplished by various methods as known in the art. Examples of such methods are described, for instance, in U.S. Pat. Nos. 5,795,587; 7,655,468; 7,993,672; 8,492,359; 8,771,728; and 8,956,572; as well as U.S. Patent Publication Nos. 2004/0262223; 2010/015218; 2012/0225129; 2012/0276209; 2012/0302622; 2013/0037977; 2013/0156845; 2014/0296322; and 2015/0209440, the contents of which are incorporated herein by reference thereto.

If desired, in some embodiments, the composition may be suited to deliver a tyrosine kinase inhibitor as a therapeutic agent. As used herein, the term “tyrosine kinase inhibitor” generally refers to a molecule, mimetic, or derivative capable of reducing, blocking, abrogating, and/or interfering with one or more biological activities, including tyrosine kinase activity. Tyrosine kinases are enzymes responsible for the activation of many proteins via signal transduction cascades. For example, tyrosine kinase inhibitors (“TKIs”) may bind to the adenosine triphosphate (ATP) binding site of VEGF resulting in blockade of intracellular signaling.

In some embodiments, the tyrosine kinase inhibitor may include, but are not limited to, an epidermal growth factor (EGF) pathway inhibitor, a platelet derived growth factor (PDGF) pathway inhibitor, a RAF-1 inhibitor, or a combination thereof. Tyrosine kinase inhibitors may include, but are not limited to, axitinib, bosutinib, cabozantinib, crizotinib, dasatinib, erlotinib, gefitinib, imatinib, lapatinib, nilotinib, pazopanib, ponatinib, ruxolitinib, sorafenib, sunitinib, vatalanib, vemurafenib, or a combination thereof. Axitinib (molecular weight of 386.5 Da), for instance, is a tyrosine kinase inhibitor that inhibits VEGF activity such as neovascularization. Thus, axitinib and other TKIs may be used for the treatment of neovascular (wet) age-related macular degeneration (AMD), the treatment of visual impairment due to diabetic macular edema (DME), the treatment of visual impairment due to macular edema secondary to retinal vein occlusion (branch RVO or central RVO), treatment of visual impairment due to choroidal neovascularization (CNV) secondary to pathologic myopia, or treatment of other retinal diseases. Cabozantinib (molecular weight of 501.5 Da), for instance, is a TKI that downregulates activation of tyrosine kinase involved in tumor angiogenesis, such as VEGF receptor.

As indicated above, therapeutic agents in the composition can include one or more antipsychotics. Antipsychotics generally refer to a class of therapeutic agents primarily used to manage and treat psychosis, such as schizophrenia. Antipsychotics are also used to treat bipolar disorder and major depressive disorder. Specifically, typical and some atypical antipsychotics are dopamine antagonists and act to impede dopamine in the brain. Further, atypical antipsychotics also influence serotonin.

Exemplary antipsychotics include both typical and atypical antipsychotics. Atypical antipsychotics that can be used herein include, but are not limited to, aripiprazole, clozapine, ziprasidone, paliperidone, risperidone, quetiapine, olanzapine, asenapine, iloperidone, lurasidone, brexpiprazole, cariprazine, and lumateperone. Salts, esters and/or isomers of antipsychotics are all meant to be encompassed in the scope of the present disclosure and shall be understood to fall under the term “antipsychotic”.

In certain embodiments, the therapeutic agent includes risperidone. Risperidone is an atypical antipsychotic and is indicated for the treatment of schizophrenia, irritability associated with autistic disorder, and as monotherapy or adjunctive therapy with lithium or valproate for the treatment of acute manic or mixed episodes associated with Bipolar 1 Disorder. Risperidone belongs to the chemical class of benzisoxazole derivatives. Risperidone has a molecular weight of 410.49 and a molecular formula of C₂₃H₂₇FN₄O₂. The structural formula of risperidone is shown below.

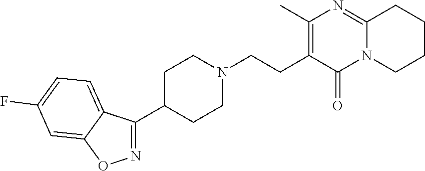 [SMILES: Cc1nc2n(c(=O)c1CCN1CCC(c3noc4cc(F)ccc34)CC1)CCCC2]

Risperidone is a monoaminergic antagonist with high affinity for the serotonin Type 2 (5HT₂), dopamine Type 2 (D₂), α1 and α2 adrenergic, and H₁ histaminergic receptors. Risperidone also shows low to moderate affinity for the serotonin 5HT_{1c}, 5HT_{1D}, 5HT_{1A} receptors and weak affinity for the dopamine D₁ and haloperidol-sensitive sigma site. Risperidone generally shows no affinity for cholinergic muscarinic or β₁ and β₃₂ adrenergic receptors.

Therapeutic agents utilized in the injectable composition can further include other therapeutic agents, such as antidepressants, which are typically co-administered with antipsychotics. Additionally, other therapeutic agents can be administered with antipsychotics as disclosed herein in order to treat or prevent side effects from the antipsychotic medication.

The therapeutic agent can include one or more GLP-1 receptor agonists. GLP-1 receptor agonists include dulaglutide, exenatide, semaglutide, liraglutide, and lixisenatide. Combinations of GLP-1 receptor agonists can also be included in the composition.

Dulaglutide is a human GLP-1 receptor agonist. The molecule is a fusion protein that includes 2 identical, disulfide-linked chains, each containing an N-terminal GLP-1 analog sequence covalently linked to the Fc portion of a modified human immunoglobulin G4 (IgG4) heavy chain by a small peptide linker. The GLP-1 analog portion of dulaglutide is 90% homologous to native human GLP-1 (7-37). Structural modifications were introduced in the GLP-1 part of the molecule responsible for interaction with the enzyme dipeptidyl-peptidase-IV (DPP-4). Additional modifications were made in an area with a potential T-cell epitope and in the areas of the IgG4 Fc part of the molecule responsible for binding the high-affinity Fc receptors and half-antibody formation. The overall molecular weight of dulaglutide is approximately 63 kilodaltons.

Dulaglutide activates the GLP-1 receptor, a membrane-bound cell-surface receptor coupled to adenylyl cyclase in pancreatic beta cells. Dulaglutide increases intracellular cyclic AMP (CAMP) in beta cells leading to glucose-dependent insulin release. Dulaglutide also decreases glucagon secretion and slows gastric emptying.

Exenatide is a synthetic peptide that was originally identified in the lizard Heloderma suspectum. Exenatide is a 39-amino acid peptide amide having the empirical formula C₁₈₄H₂₈₂N₅₀O₆₀S and molecular weight of 4186.6 Daltons. The amino acid sequence of exenatide partially overlaps that of human GLP-1. Exenatide has been shown to bind and activate the human GLP-1 receptor in vitro. Such binding leads to an increase in both glucose-dependent synthesis of insulin and in vivo secretion of insulin from pancreatic beta cells, by mechanisms involving cyclic AMP and/or other intracellular signaling pathways. Exenatide promotes insulin release from pancreatic beta cells in the presence of elevated glucose concentrations.

Semaglutide is a human GLP-1 receptor agonist. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by DPP-4. A minor modification is also made in position 34 to ensure the attachment of only one fatty di-acid. The molecular formula of semaglutide is C₁₈₇H₂₉₁N₄₅O₅₉ and the molecular weight is 4113.58 g/mol.

Liraglutide is an analog of human GLP-1 and acts as a GLP-1 receptor agonist. The peptide precursor of liraglutide has been engineered to be 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. The molecular formula of liraglutide is C₁₇₂H₂₆₅N₄₃O₅₁ and the molecular weight is 3751.2 Daltons.

Lixisenatide is modified from exendin-4 and is a 44-amino-acid peptide with the C-terminal Pro replaced by 6 Lys residues. Its molecular weight is 4858.5 and its molecular formula is C₂₁₅H₃₄₇N₆₁O₆₅S. In vitro studies found that lixisenatide has four times greater affinity for the GLP-1 receptor than native GLP-1 and exenatide.

In certain other embodiments, the GLP-1 receptor agonist can include molecules that are selective for the human GLP-1 receptor. For instance, the GLP-1 receptor agonist can include molecules, compounds, metabolites, and/or isomers that are selective for the GLP-1 receptor. For instance, in certain embodiments the GLP-1 receptor agonist can include small molecules or peptide fragments. Additionally, in certain embodiments native GLP-1 can be utilized in the injectable composition disclosed herein. In such embodiments, the native GLP-1 can be formulated with one or more excipients in order to increase the half live of GLP-1 in vivo.

### D. Excipients

The composition may also optionally contain one or more additives or excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, dispersants, other types of antimicrobial agents, preservatives, etc. to enhance properties and processability. When employed, the optional excipient(s) typically constitute from about 0.01 wt. % to about 30 wt. %, and in some embodiments, from about 0.05 wt. % to about 15 wt. %, and in some embodiments, from about 0.1 wt. % to about 10 wt. % of the composition. Other known antimicrobial agents and/or preservatives may also be employed to help prevent surface growth and attachment of bacteria, such as metal compounds (e.g., silver, copper, or zinc), metal salts, quaternary ammonium compounds, etc.

To help further control the release of the therapeutic agent(s), a one or more hydrophilic compounds may also be incorporated into the composition that are soluble and/or swellable in water. The use of such hydrophilic compounds can accelerate therapeutic agent release from the in situ implant formed from the composition. For instance, the use of hydrophilic compounds can lead to higher burst rates of the therapeutic agent from the composition. When employed, the weight ratio of the POE polymer(s) the hydrophilic compounds within the composition may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 65 wt. %, in some embodiments from about 2 wt. % to about 50 wt. %, and in some embodiments, from about 5 wt. % to about 40 wt. % of the composition, while POE polymer(s) typically constitute from about 40 wt. % to about 99 wt. %, in some embodiments from about 50 wt. % to about 98 wt. %, and in some embodiments, from about 60 wt. % to about 95 wt. % of the composition.

Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc. Examples of sugar alcohols include sugar alcohols (e.g., xylitol, sorbitol, mannitol, maltitol, erythritol, galactitol, isomalt, inositol, lactitol, etc.). In embodiments, the composition includes one or more sugar alcohols (e.g., mannitol).

In one embodiment, the hydrophilic compound can include polyvinylpyrrolidone (PVP). PVP can be included in the composition and can function as a dispersant or wetting agent. As used herein “dispersants” can refer to one or more compounds used to facilitate dispersion of the therapeutic agent in the solvent and polymer system. Notably, the dispersant can facilitate solubilizing of the therapeutic agent to facilitate dispersion within the solvent/polymer system.

To help further control the release of the therapeutic agent(s), a one or more hydrophobic compounds may also be incorporated into the composition that are insoluble in water. Addition of such amphiphilic or hydrophobic compounds can reduce burst release of the therapeutic agent from the in situ implant and can modify the overall morphology of the in situ implant formed from the injectable composition. For instance, inclusion of one or more hydrophobic agents can produce an in situ implant having a porous or sponge-like structure. Inclusion of one or more hydrophobic agents can also facilitate retention of the solvent in the in situ implant, which can decrease release of the therapeutic agent from the in situ implant formed from the injectable composition.

When employed, the weight ratio of the POE polymer(s) the hydrophobic compounds within the composition may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophobic compounds may, for example, constitute from about 1 wt. % to about 65 wt. %, in some embodiments from about 2 wt. % to about 40 wt. %, and in some embodiments, from about 5 wt. % to about 20 wt. % of the composition. Suitable hydrophobic compounds include lipids, glycolipids, lipid derivatives, lipid analogues, cholesterol, oils, fats, waxes, sterols, monoglycerides, diglycerides, triglycerides, phospholipids, and mixtures thereof.

In embodiments, pH adjusting additives can also be utilized in the injectable composition. For instance, in order to facilitate faster surface erosion from the composition, one or more acids can be incorporated into the injectable composition that can catalyze hydrolysis of the POE polymer upon injection. Suitable acids can include any acid that is biocompatible with the POE polymer and therapeutic agent. Suitable acids include hydrochloric acid, citric acid, acetic acid, nitric acid, carboxylic acids (e.g., maleic acid), and combinations thereof. Similarly, in other embodiments, to slow release of the therapeutic agent from the composition one or more bases can be included in the composition. Suitable bases can include Arrhenius bases, Bronsted bases, Lewis bases, and combinations thereof. The bases included can be strong or weak bases. Suitable bases include hydroxides (e.g., sodium hydroxide, potassium hydroxide, aluminum hydroxide), ammonia, bicarbonates (e.g., sodium bicarbonate, sodium carbonate), alanine, methylamine, ammonium hydroxide, and combinations thereof.

### II. Use of the Injectable Flowable Composition

The injectable flowable composition can be formulated as a viscous liquid or gel having a viscosity such that it can be injected from a standard hypodermic syringe, a catheter or a trocar, that has been pre-filled with the injectable flowable composition containing the therapeutic agent. In embodiments, injection of the composition takes place utilizing the smallest size needle (e.g., smallest diameter) or catheter to reduce discomfort to the patient during injection. The injection can be subcutaneous, intramuscular, intravascular (high/low flow), intramyocardial, adventitial, intratumoral, or intracerebral. The composition can be injected at wound sites, tight joint spaces or in the body cavity of a human or animal. The composition can be injected through a needle or a catheter ranging from 16 gauge and higher, preferably 18 gauge or higher, preferably 20 gauge and higher, more preferably 22 gauge and higher, even more preferably 24 gauge and higher.

The injectable flowable composition can be effective for sustained release of a therapeutic agent over a prolonged period of time. Of course, the actual dosage level of the therapeutic agent delivered will vary depending on the particular agent employed and the time period for which it is intended to be released. The dosage level is generally high enough to provide a therapeutically effective amount of the therapeutic agent to render a desired therapeutic outcome, i.e., a level or amount effective to reduce or alleviate symptoms of the condition for which it is administered. More particularly, a used herein, the phrase “therapeutically effective amount” means a dose of the therapeutic agent that results in a detectable improvement in one or more symptoms of a disorder, or a dose of antibody that inhibits, prevents, lessens, or delays the progression of a disorder. The exact amount necessary will vary, depending on the subject being treated, the age and general condition of the subject to which the antibody is to be delivered, the capacity of the subject's immune system, the degree of effect desired, the severity of the condition being treated, the particular antibody selected and mode of administration of the composition, among other factors. In one embodiment, for example, a therapeutically effective amount can be from about 0.001 mg to about 10 mg per day, in some embodiments from about 0.01 mg to about 9 mg per day, in some embodiments from about 0.1 to about 8 mg per day, in some embodiments from about 1 mg to about 7 mg per day, in some embodiments from about 2 mg to about 6 mg per day, in some embodiments, from about 3 mg to about 5 mg per day.

The amount of the therapeutic agent contained within the individual doses may be expressed in terms of milligrams of drug per kilogram of patient body weight (i.e., mg/kg). For example, the therapeutic agent may be administered to a patient at a dose of about 0.0001 to about 10 mg/kg of patient body weight.

The injectable flowable composition(s) can be loaded with from about 5 mg to about 300 mg of one or more therapeutic agents, such as from about 75 mg to about 275 mg, such as from about 100 mg to about 225 mg, such as from about 125 mg to about 200 mg, such as from about 25 mg to about 215 mg. Certain injectable compositions can also be loaded with from about 500 mg to about 2000 mg of one or more therapeutic agents, such as from about 600 mg to about 1800 mg, such as from about 800 mg to about 1600 mg, such as from about 1,000 mg to about 1,500 mg. For instance, the implant can be loaded with from about 600 mg to about 900 mg, such as from about 700 mg to about 800 mg. Additionally, the amount of therapeutic agent loaded into the injectable composition can be modified (e.g., increased and/or decreased) depending on the amount of implantation time desired or route of implantation (e.g., subcutaneously vs. intramuscular).

The volume of injectable flowable composition injected into the patient can vary and ranges from about 0.5 mL up to about 10 mL per, such as 1 mL to about 9 mL, such as about 2 mL to about 8 mL of composition per injections. Depending on the therapeutic agent utilized and the disease or condition treated, multiple injections of the injectable flowable composition are contemplated herein.

The injectable flowable composition can be injected into various locations within the patient, for example, subcutaneously, orally, mucosally, etc., using standard techniques. The delivery route may be intrapulmonary, gastroenteral, subcutaneous, intramuscular, or for introduction into the central nervous system (e.g., intrathecal, intracranial, intraventricular), intraperitoneum or for intraorgan delivery. The composition can be injected in, on, adjacent to, or near a tumor, such as a tumor of the pancreas, biliary system, gallbladder, liver, small bowel, colon, brain, lung, eye, etc.

The injectable flowable composition of the present disclosure can also be injected during surgical procedures, such as via a laparoscopic procedure or robotic surgery, such as guided robotic surgery. The manner in which the injectable composition is injected within a patient may vary as known to those skilled in the art. The route of injection can include intravenous (IV), intramuscular (IM), and combinations thereof. The route of injection of the injectable composition can depend on a variety of factors, such as the disease/condition being treated, whether surgery is required, metastasis, tumor size, tumor size, tumor type, patient age, physical condition, fertility status, and any other requirements.

The injectable composition can be effective for sustained release of one or more therapeutic agents over a prolonged period of time. For example, the injectable composition can release therapeutic agent for a time period of about 3 days or more, in some embodiments, 5 days or more, in some embodiments about 10 days or more, in some embodiments from about 20 days to about 365 days, and in some embodiments, from about 30 days to about 180 days. Further, the present inventors have also discovered that a therapeutic agent can be released in a highly controlled manner over the course of the release time period. After a time period of 15 days, for example, the cumulative weight-based release ratio of a therapeutic agent may be from about 10% to about 100%, such as from about 20% to about 90%, such as from about 30% to about 80%, such as from about 40% to about 50%. Likewise, after a time period of 35 days, the cumulative weight-based release ratio of a therapeutic agent may be from about 20% to about 100%, in some embodiments from about 30% to about 90%, in some embodiments from about 40% to about 80%, such as from about 50% to about 70%, and in some embodiments, from about 35% to about 50%. The “cumulative weight-based release ratio” may be determined by dividing the total amount of therapeutic agent released at a particular time interval by the total amount of the therapeutic agent initially present, and then multiplying this number by 100.

The injectable composition may be suitable for delivering a therapeutic agent to treat a wide variety of conditions, such as cancer, allergies, inflammation, immunologically mediated diseases, metabolic diseases, eye disorders (e.g., angiogenic eye disorders), etc. In one embodiment, the injectable composition may release a therapeutic agent that can treat cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In one specific embodiment, the injectable composition may deliver an anti-HER2 antibody to treat a cancer that “overexpresses” a HER receptor. Such cancers include those that have significantly higher levels of a HER receptor, such as HER2, at the cell surface thereof, compared to a noncancerous cell of the same tissue type. Such overexpression may be caused by gene amplification or by increased transcription or translation.

The injectable composition may also be suitable for treatment of an angiogenic eye disorder, which includes any disease of the eye which is caused by or associated with the growth or proliferation of blood vessels or by blood vessel leakage. Non-limiting examples of angiogenic eye disorders that are treatable using the methods of the present disclosure include age-related macular degeneration (e.g., wet AMD, exudative AMD, etc.), retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies.

The injectable flowable composition can be used to treat a variety of psychotic disorders including psychosis, schizophrenia, schizoaffective disorder, schizophrenium disorder, brief psychotic disorder, delusional disorder, shared psychotic disorder, substance-induced psychotic disorder, and paraphrenia. Depending on the route of administration for delivery of the injectable composition, the amount of antipsychotic present in the injectable composition can vary.

As noted above, the injectable composition may be particularly suitable for delivering a GLP-1 receptor agonist for the treatment of diabetes (e.g., type 2 diabetes) or other approved uses for GLP-1 receptor agonists. The injectable composition may also be employed together with other therapies for the treatment of diabetes, including insulin injections and/or metformin administration. In such embodiments, these additional therapeutic agents can be administered to the patient in a variety of dosage forms, including, oral dosage forms, intravenous dosage forms, subcutaneous dosage forms, including depot injections, hydrogel injections, intramuscular injections, etc., or intravaginal dosage forms. Additional therapeutic agents can be administered via any suitable route and can be used in combination with the injectable composition disclosed herein. The injectable composition may also be particularly suitable for treating metabolic syndrome, atherosclerosis, dyslipidemia, obesity, and/or adipositas.

If desired, the injectable composition can be sealed within a package or a vial prior to use. For instance, components of the injectable composition can be stored in a vial and the desired solvent can be injected into the vial just prior to injection. The components are then mixed with the solvent in the vial forming a viscous liquid that can then be administered via a syringe. For instance, the vial can contain the hydrophobic polymer and therapeutic agent in dry or crystallized form. Upon injection of the solvent and mixing, a viscous composition (e.g., a gel-like composition) is formed that can be administered to the patient.

The POE polymer, solvent, and therapeutic agent can be formulated in a variety of manners in order to form a suitable injectable flowable composition that can be injected. In embodiments, the injectable composition can be prepared by mixing or blending the POE polymer, solvent and therapeutic agent(s). Such blending is carried out in a manner suitable to obtain a homogeneous distribution of the components throughout the composition, by mixing the components in any order necessary to achieve such homogeneity. The mixing or blending can be performed by any method and generally at temperatures less than about 50° C., such as less than about 40° C., such as generally at room temperatures (e.g., 20° C. to 22° C.). For instance, the desired amount of POE polymer and therapeutic agent can be mixed with differing amounts of solvent as disclosed herein. Upon blending, a viscous liquid composition (e.g., a gel) is formed. The injectable flowable composition can then be administered to a patient via methods disclosed hereinabove.

As noted, the amount of POE polymer, solvent, and therapeutic agent can be selected such that the therapeutic agent is homogenously dispersed throughout the POE polymer. In embodiments, discrete domains of therapeutic agent in solid crystalline form can be dispersed throughout the POE polymer. During injection, an in situ implant is formed having a POE polymer matrix with the therapeutic agent dispersed therein as the solvent diffuses from the injectable composition into the surrounding extracellular fluid. Upon surface erosion of the polymer matrix, the discrete domains of therapeutic agent can be released from the polymer matrix to provide therapeutically effective amounts of the therapeutic agent. In other embodiments, however, the therapeutic agent may be completely miscible within the POE polymer and solvent and, thus, is dispersed as a solution throughout the POE polymer matrix upon injection. It should be appreciated, that depending on the solubility and/or particle size of the desired therapeutic agent, a variety of dispersions including coarse dispersions (suspensions), colloidal dispersions, or molecular dispersions (e.g., true solution or liquid) can be achieved.

In other embodiments, the POE polymer can be blended with the therapeutic agent and a solvent (e.g., water) to form a solution containing the POE polymer and therapeutic agent. The resultant solution can then be subjected to a lyophilization or freeze-drying process to produce a solid powder material containing a blend of POE polymer and therapeutic agent. This solid powder blend can be stored in a suitable container (e.g., a vial) until it is ready for use. To use, the desired amount of solvent is mixed with the solid powder blend of POE polymer and therapeutic agent to reconstitute the powder blend to form an injectable flowable composition. Notably, reconstitution of the powder blend via the solvent can take place just before administration (e.g., injection) of the composition.

### Examples 1-5

POE formulations were prepared using CHDM (MW 49 KDa), CHDM-GL, and/or TED (MW 27 KDa). These polymer grade(s) were dissolved in NMP to yield a POE solution in NMP. Risperidone was then added to yield a suspension. The total volume of NMP used in all formulations was 2.5 mL. A total of five formulations were prepared. Tables 1 and 2 show the formulation percentages (5) with and without NMP, as indicated.

TABLE 1

| | Comparative | Example | Example | Example | Example |
|---|---|---|---|---|---|
| Ingredients | Example 1 | 2 | 3 | 4 | 5 |
| CHDM | 70.6 | 52.8 | 35.2 | 17.4 | 0 |
| (% w/w) | | | | | |
| CHDM-GL | 0 | 17.9 | 35.2 | 53.1 | 0 |
| (% w/w) | | | | | |
| TEG (% w/w) | 0 | 0 | 0 | 0 | 70.4 |
| Risperidone | 29.4 | 29.3 | 29.6 | 29.5 | 29.6 |
| (% w/w) | | | | | |

TABLE 2

| | Comparative | Example | Example | Example | Example |
|---|---|---|---|---|---|
| Ingredients | Example 1 | 2 | 3 | 4 | 5 |
| CHDM | 22.3 | 16.7 | 11.1 | 5.5 | 0 |
| (% w/w) | | | | | |
| CHDM-GL | 0 | 5.6 | 11.1 | 16.7 | 0 |
| (% w/w) | | | | | |
| TEG (% w/w) | 0 | 0 | 0 | 0 | 22.2 |
| Risperidone | 9.3 | 9.3 | 9.3 | 9.3 | 9.3 |
| (% w/w) | | | | | |
| NMP (% w/w) | 68.4 | 68.4 | 68.5 | 68.4 | 68.5 |

The total weights of each component in each formulation are summarized in Table 3. For instance, Table 3 demonstrates the weight of each polymer, either CHDM, CHDM-GL, TEG, or a combination thereof, the weight of the active ingredient, Risperidone, and the weight of the solvent, NPM, for each of the five formulations all measured in mg.

TABLE 3

| | Comparative | Example | Example | Example | Example |
|---|---|---|---|---|---|
| Ingredients | Example 1 | 2 | 3 | 4 | 5 |
| CHDM (mg) | 839.7 | 627.5 | 416.3 | 206.3 | 0 |
| CHDM-GL | 0 | 212.5 | 415.4 | 629.9 | 0 |
| (mg) | | | | | |
| TEG (mg) | 0 | 0 | 0 | 0 | 832.6 |
| Risperidone | 349.6 | 348.9 | 349.8 | 350.7 | 350 |
| (mg) | | | | | |
| NMP (mg) | 2575 | 2575 | 2575 | 2575 | 2575 |

The total weights of each component in each 0.5 mL injection are summarized in Table 4. For instance, Table 4 demonstrates the weight of each polymer, either CHDM, CHDM-GL, TEG, or a combination thereof, the weight of the active ingredient, Risperidone, and the weight of the solvent, NPM, for each injection of each of the five formulations all measured in mg.

TABLE 4

| | Comparative | Example | Example | Example | Example |
|---|---|---|---|---|---|
| Ingredients | Example 1 | 2 | 3 | 4 | 5 |
| CHDM (mg) | 135.1 | 97.3 | 66 | 33.4 | 0 |
| CHDM-GL | 0 | 33 | 65.9 | 102.1 | 0 |
| (mg) | | | | | |
| TEG (mg) | 0 | 0 | 0 | 0 | 132.7 |
| Risperidone | | | | | |
| (mg) | 56.3 | 54.1 | 55.5 | 56.8 | 55.8 |
| NMP (mg) | 414.4 | 399.3 | 408.4 | 417.3 | 410.3 |
| Total | 605.8 | 583.7 | 595.8 | 609.6 | 598.8 |
| formulation | | | | | |
| mass with | | | | | |
| NMP | | | | | |
| (mg) | | | | | |

The total weights of each component in each 0.5 mL injection are summarized in Table 5 excluding the NMP. For instance, Table 5 demonstrates the weight of each polymer, either CHDM, CHDM-GL, TEG, or a combination thereof, the weight of the active ingredient, Risperidone, for each injection of each of the five formulations all measured in mg.

TABLE 5

| | Comparative | Example | Example | Example | Example |
|---|---|---|---|---|---|
| Ingredients | Example 1 | 2 | 3 | 4 | 5 |
| CHDM (mg) | 135.1 | 97.3 | 66 | 33.4 | 0 |
| CHDM-GL | 0 | 33 | 65.9 | 102.1 | 0 |
| (mg) | | | | | |
| TEG (mg) | 0 | 0 | 0 | 0 | 132.7 |
| Risperidone | 56.3 | 54.1 | 55.5 | 56.8 | 55.8 |
| (mg) | | | | | |
| NMP (mg) | 414.4 | 399.3 | 408.4 | 417.3 | 410.3 |
| Total | 605.8 | 583.7 | 595.8 | 609.6 | 598.8 |
| formulation | | | | | |
| mass with | | | | | |
| NMP | | | | | |
| (mg) | | | | | |

The weight ratios of polymer to polymer and polymer to drug are summarized in Table 6.

TABLE 6

| | Comparative | Example | Example | Example | Example |
|---|---|---|---|---|---|
| Ratio | Example 1 | 2 | 3 | 4 | 5 |
| CHDM/CHDM-GL | CHDM only | 2.95 | 1 | 0.33 | TEG |
| ratio (w/w) | | | | | only |
| Polymer to | 2.40 | 2.41 | 2.38 | 2.38 | 2.38 |
| drug ratio (w/w) | | | | | |

For risperidone release, 0.5 mL of each of Examples 1-5 were individually injected into 1 mL of Phosphate Buffer Solution (PBS) with a pH of 7.4 in a dialysis bag made of regenerated cellulose with a MW cut off of 15 KDa. Each dialysis bag was into a glass tube containing an addition 50 mL of PBS with a pH of 7.4. Each glass tube was then placed into an incubator at 37° C. and shook at 100 rpm. At regular intervals, the PBS in the glass tubes was replaced with fresh PBS.

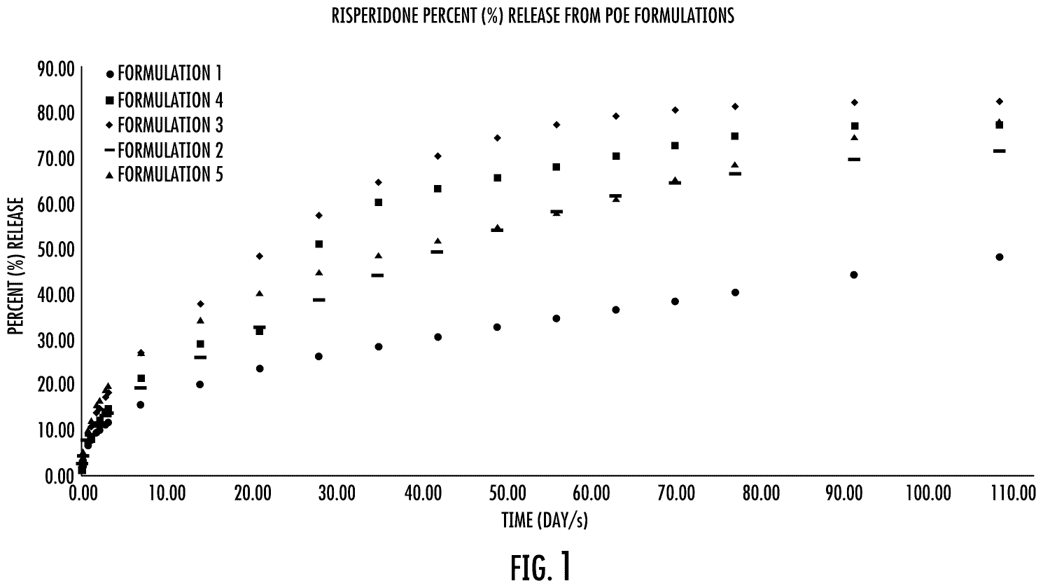

Drug release data for each of the five formulations is summarized in FIG. 1, Table 7, and Table 8. For instance, FIG. 1 depicts the percentage of Risperidone released from the POE formulations over the course of 63 days. These data points are further summarized in Table 7 which demonstrates the average percentage of drug released across 63 days. Table 8 demonstrates the average cumulative weight in mg of the drug released from each formulation across 63 days.

TABLE 7

| | Average percent (w/w %) drug release | | | | |
|---|---|---|---|---|---|
| Time | Comparative | Example | Example | Example | Example |
| (day/s) | Example 1 | 2 | 3 | 4 | 5 |
| 0.04 | 2.34 | 2.76 | 2.87 | 1.29 | 1.80 |
| 0.17 | 3.54 | 4.40 | 4.73 | 2.55 | 5.26 |
| 0.75 | 6.82 | 7.89 | 9.23 | 7.10 | 10.15 |
| 1.13 | 7.89 | 9.04 | 10.86 | 8.65 | 12.15 |
| 1.75 | 9.62 | 11.08 | 13.99 | 11.45 | 15.71 |
| 2.13 | 10.07 | 11.68 | 14.93 | 12.21 | 16.68 |
| 2.79 | 11.27 | 13.23 | 17.34 | 14.09 | 19.02 |
| 3.13 | 11.73 | 13.85 | 18.34 | 14.85 | 19.95 |
| 7 | 15.78 | 19.45 | 27.21 | 21.49 | 27.23 |
| 14 | 20.16 | 26.11 | 37.97 | 29.07 | 34.34 |
| 21 | 23.67 | 32.79 | 48.48 | 31.86 | 40.33 |
| 28 | 26.38 | 38.80 | 57.44 | 51.10 | 45.05 |
| 35 | 28.54 | 44.20 | 64.72 | 60.23 | 48.70 |
| 42 | 30.61 | 49.33 | 70.49 | 63.24 | 51.96 |
| 49 | 32.77 | 54.16 | 74.57 | 65.68 | 54.83 |
| 56 | 34.67 | 58.20 | 77.48 | 68.07 | 58.02 |
| 63 | 36.63 | 61.79 | 79.42 | 70.49 | 61.09 |
| 70 | 38.54 | 64.57 | 80.54 | 72.76 | 65.04 |
| 77 | 40.36 | 66.65 | 81.27 | 74.68 | 68.66 |
| 91 | 44.26 | 69.72 | 82.13 | 77.04 | 74.45 |
| 108 | 48.02 | 71.66 | 82.36 | 77.32 | 77.85 |

TABLE 8

| | Average cumulative drug (mg) release | | | | |
|---|---|---|---|---|---|
| Time | Comparative | Example | Example | Example | Example |
| (day/s) | Example 1 | 2 | 3 | 4 | 5 |
| 0.04 | 1.31 | 1.54 | 1.50 | 0.69 | 1.00 |
| 0.17 | 1.98 | 2.46 | 2.46 | 1.37 | 2.91 |
| 0.75 | 3.82 | 4.41 | 4.81 | 3.81 | 5.61 |
| 1.13 | 4.42 | 5.06 | 5.66 | 4.64 | 6.72 |
| 1.75 | 5.38 | 6.19 | 7.29 | 6.14 | 8.68 |
| 2.13 | 5.63 | 6.53 | 7.78 | 6.56 | 9.22 |
| 2.79 | 6.31 | 7.40 | 9.03 | 7.57 | 10.52 |
| 3.13 | 6.57 | 7.74 | 9.55 | 7.97 | 11.03 |
| 7 | 8.83 | 10.87 | 14.17 | 11.54 | 15.06 |
| 14 | 11.28 | 14.60 | 19.78 | 15.61 | 18.99 |
| 21 | 13.25 | 18.33 | 25.26 | 17.10 | 22.30 |
| 28 | 14.76 | 21.69 | 29.93 | 27.43 | 24.91 |
| 35 | 15.97 | 24.71 | 33.72 | 32.33 | 26.93 |
| 42 | 17.13 | 27.58 | 36.72 | 33.95 | 28.73 |
| 49 | 18.34 | 30.28 | 38.85 | 35.26 | 30.32 |
| 56 | 19.40 | 32.54 | 40.36 | 36.54 | 32.08 |
| 63 | 20.50 | 34.54 | 41.37 | 37.84 | 33.78 |
| 70 | 21.57 | 36.10 | 41.96 | 39.06 | 35.96 |
| 77 | 22.59 | 37.27 | 42.34 | 40.09 | 37.96 |
| 91 | 24.77 | 38.98 | 42.79 | 41.36 | 41.16 |
| 108 | 26.88 | 40.06 | 42.91 | 41.51 | 43.04 |

### Example 6

Example 6 demonstrates the degradation kinetics of TEG containing POE polymers containing no therapeutic agent.

A polyorthoester polymer produced from the polymerization of DETOSU and triethylene glycol with a weight average molecular weight of 27 kDa (TEG-27) was placed in phosphate buffered saline solution with a pH of 7.4 and held at 37° C. At specified time intervals, it was recovered from the solution, dried with a chem wipe, and weighed, before being placed back in the buffer solution. The study was halted when material had broken down to the point where it no longer could be accurately recovered from the vial and weighed.

The polymer exhibited essentially no weight loss for the first week of the study, before rapidly breaking down between day 8 and day 17. After day 17, very little material remained, and the study was ended. The weight loss percentage as a function of time is shown in the table below. This polymer degrades so rapidly, suggesting that it would not be useful for a long-acting drug delivery system targeting drug release for more than 2-3 weeks.

TABLE 9

| Time | TEG-27 Weight |
|---|---|
| (days) | Loss |
| 1 | 0% |
| 2 | 0% |
| 3 | 0% |
| 4 | 0% |
| 8 | 0% |
| 14 | 55% |
| 17 | 94% |

### Examples 7-12

Examples 7-12 demonstrate the degradation kinetics of CHDM containing POE polymers containing no therapeutic agent.

Two polyorthoester polymers were used to prepare the samples used in this example. The first polymer, given the nomenclature of CHDM-32, was produced from the polymerization of DETOSU and CHDM, and has a weight average molecular weight of 32 kDa. The second polymer, given the nomenclature of CGL-39, was produced from the polymerization of DETOSU and CHDM-glycolide, and has a weight average molecular weight of 39 kDa.

Blends of these two polymers were produced by weighing each polymer in a vial, codissolving the polymers in methylene chloride under constant stirring, and then evaporating the methylene chloride first at ambient conditions followed by under vacuum.

Rod-shaped test specimens with the nominal dimensions of 2 mm diameter×10 mm long were made from pure polymers as well as the above described blends. All specimens were produced using a vacuum compression molding apparatus (MeltPrep® VCM).

All rod-shaped test specimens were placed in phosphate buffered saline solution with a pH of 7.4 and held at 37° C. At specified time intervals, specimens were recovered from the solution, dried with a chem wipe, and weighed, before being placed back in the buffer solution. Testing of a specimen was halted when material had broken down to the point where it no longer could be accurately recovered from the vial and weighed.

The weight loss of each polymer or polymer blend as a function of time are shown in the table below. The data indicates that increasing the quantity of CHDM-glycolide included in the blend accelerates the degradation of the polymer. Polymer without the CHDM-glycolide monomer (Pure CHDM-32) shows very little degradation, whereas polymers with high levels of CHDM-glycolide (25% CHDM-32/75% CGL-39, and Pure CGI-39) degrade completely within a few days. It is notable that these compositions containing high levels of glycolide-modified polymers degrade so rapidly, they would be impractical for developing a long-acting drug delivery system. Table 10 illustrates the weight loss as a function of time for CHDM-32, CGL-39, and their blends.

TABLE 10

| | Ex. 7 | Ex. 8 | Ex. 9 | Ex. 10 | Ex. 11 | Ex. 12 |
|---|---|---|---|---|---|---|
| | Pure | 90% CHDM-32/ | 75% CHDM-32/ | 50% CHDM-32/ | 25% CHDM-32/ | Pure |
| Days | CHDM-32 | 10% CGL-39 | 25% CGL-39 | 50% CGL-39 | 75% CGL-39 | CGL-39 |
| 1 | 0% | 0% | 0% | 10% | 95% | 14% |
| 2 | 0% | 0% | 0% | 30% | | 57% |
| 3 | 0% | 0% | 1% | 47% | | 88% |
| 4 | 0% | 0% | 4% | 56% | | |
| 7 | 0% | 1% | 19% | 62% | | |
| 9 | 0% | 6% | 41% | 64% | | |
| 11 | 0% | 14% | 46% | 67% | | |
| 15 | 0% | 25% | 51% | 69% | | |
| 18 | 0% | 30% | 57% | 70% | | |
| 21 | 1% | 34% | 61% | 71% | | |
| 25 | 1% | 40% | 64% | 73% | | |
| 31 | 1% | 51% | 70% | 77% | | |
| 38 | 3% | 58% | 74% | 81% | | |
| 42 | 3% | 64% | 79% | 85% | | |
| 52 | 3% | 68% | 81% | 87% | | |
| 59 | 4% | 72% | 85% | 90% | | |
| 66 | 6% | 76% | 87% | 91% | | |
| 70 | 6% | 78% | 89% | 92% | | |
| 77 | 8% | 82% | | | | |

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only and is not intended to limit the disclosure so further described in such appended claims.

What is claimed is:

1. An injectable flowable composition for delivery of one or more therapeutic agents, comprising:
one or more polymers comprising a first poly ortho ester (POE) polymer wherein the first POE polymer has a T_{g} of between about −10° C. and 110° C. as determined in accordance with ASTM E1640-18;
one or more solvents; and
one or more therapeutic agents, wherein the one or more agents comprises a basic therapeutic agent.

2. The injectable gel composition of claim 1, wherein the one or more therapeutic agents constitute from about 40 wt. % to about 70 wt. % of the composition.

3. The injectable gel composition of claim 1, wherein the POE polymer has an acetal monomer content of from about 20 mol. % to about 80 mol. %.

4. The injectable gel composition of claim 1, wherein the POE polymer has a polyol monomer content of from about 20 mol. % to about 80 mol. %.

5. The injectable gel composition of claim 4, wherein the polyol monomer content comprises from about 1 mol. % to about 60 mol. % of one or more modified polyols.

6. The injectable gel composition of claim 1, wherein the POE polymer has a weight-average molecular weight of from about 1 kDa to about 100 kDa.

7. The injectable gel composition of claim 1, wherein the first POE polymer has a T_{g} of between about −10° C. and 50° C. as determined in accordance with ASTM E1640-18.

8. The injectable gel composition of claim 1, wherein the solvent comprises an organic solvent.

9. The injectable gel composition of claim 1, wherein the organic solvent comprises N-methyl-2-pyrrolidone (NMP), 2-pyrrolidone, dimethyl sulfoxide (DMSO), benzyl benzoate, benzyl alcohol, triacetin, glycofurol, polyethylene glycol, ethyl acetate, or mixtures thereof.

10. The injectable gel composition of claim 9, wherein the organic solvent comprises NMP.

11. The injectable gel composition of claim 1, wherein the one or more therapeutic agents comprise one or more antipsychotics.

12. The injectable gel composition of claim 11, wherein the one or more antipsychotics comprise risperidone, olanzapine, quetiapine, ziprasidone, aripiprazole, and functional analogues thereof.

13. The injectable gel composition of claim 1, wherein the therapeutic agent is in crystalline form.

14. The injectable gel composition of claim 1, comprising a second POE polymer.

15. The injectable gel composition of claim 14, wherein the second POE polymer has a glass transition temperature (T_{g}) that is greater than the first POE polymer.

16. The injectable gel composition of claim 1, wherein the composition forms an in situ implant upon injection.

17. A method for prohibiting and/or treating a condition, disease, and/or cosmetic state of a patient in need thereof, the method comprising injecting the composition of claim 1 in the patient.
